(19) United States
(12) Patent Application Publication
(10) Pub. No.: US 2025/0216352 A1
(43) Pub. Date: Jul. 3, 2025

(54) LEAF-ATTACHABLE SENSOR FOR CELL WATER ANALYSIS BY ELECTROCHEMICAL IMPEDANCE SPECTROSCOPY AND METHOD FOR PRODUCING SAME

(71) Applicant: CENTRO NACIONAL DE PESQUISA EM ENERGIA E MATERIAIS, Campinas (BR)

(72) Inventors: Renato Sousa LIMA, Campinas (BR); Julia Adorno BARBOSA, Campinas (BR); Angelo Luiz GOBBI, Campinas (BR)

(21) Appl. No.: 18/866,848

(22) PCT Filed: May 17, 2023
(86) PCT No.: PCT/BR2023/050149

§ 371 Date: Nov. 18, 2024

(30) Foreign Application Priority Data
May 18, 2022 (BR) 1020220097283

(51) Int. Cl. G01N 27/02; G01N 33/00; G01N 33/483

(52) U.S. Cl. CPC G01N 27/026; G01N 33/0098; G01N 33/4833

(57) ABSTRACT

The present description refers to a sensor comprising stand-alone Ni electrodes (1) formed by film with a thickness between 30 and 50 μm, attachable to leaves by fixing means (2) made of flexible, porous and transparent material. Signal reading and actuation means (5) for carrying out electrochemical impedance spectroscopy tests are provided in electrical connection with the Ni electrodes (1). The production process of this sensor is also described, involving well-established, scalable and reproducible techniques, such as photolithography and electroplating.

### FIELD OF THE INVENTION

The present description is from the field of materials analysis by impedance investigation, specifically from the field of sensing cellular water content in plant leaves in vivo and in situ.

### BACKGROUND OF THE INVENTION

Quantitative measurements of the extent of leaf hydration can provide crucial information about the effectiveness of plant irrigation, as leaf water content can serve as a marker for plant physiological health. Impedimetric wearable sensors are promising tools for determining leaf water loss (abbreviated here by the English acronym LWL), as they enable real-time and in situ for rapid decision management in agriculture and for kinetic assessment in studies, for example, on the toxicity of nanomaterials and the efficiency of fertilizers. However, the production of these sensors for impedance investigation has been limited by the difficulty of producing appropriate electrodes on a commercial scale. Furthermore, these applications involve long-term tests that imply ongoing challenges, such as the biocompatibility of the electrodes and losses in their performance in the face of climate fluctuations.

### STATE OF ART

Recently, wearable electrodes have also been integrated into leaves for the indoor analysis of physiological parameters (water loss and growth rate; (ACS Omega, 4(5), 2019; npj Flexible Electron, 2(24), 2018; Sci. Adv, 463, 2019) and abiotic microclimate factors, such as humidity, temperature and sunlight intensity (ACS Omega, 4(5), 2019; npj Flexible Electron. 2(24), 2018). The biggest challenges for this area are adherence to the leaf surface, biocompatibility and sensor sensitivity for continuous analysis of subtle physiological changes in response to external stimuli. The risks of sensor delamination, with loss of conformal contact between electrodes and leaf, and interference with the biological functions of plants become particularly greater due to the requirement for long-term tests (weeks to months). It is also worth highlighting the existence of thousands of microstructured leaf topography and the simultaneous variation of two or more physiological parameters depending on a single stress factor.

Briefly, Zhao and colleagues (ACS Omega, 4(5), 2019) analyzed physiological and microclimate parameters through the integration of 4 sensors in leaves, namely, impedance sensor (water content), voltage sensor (growth rate), thermoresistor (temperature) and phototransistor (light). These devices consisted of patterns of metallic films (copper and carbon nanotubes) on flexible polyimide (PI) films. The electrodes were manufactured by a conventional photolithographic method, based on spinning, physical mask-assisted pattern transfer, liquid phase development, vapor phase metal deposition and lift-off. The total thickness of the device was 8 μm. As limitations, reproducibility tests, long-term biocompatibility of electrodes and quantification of water content and leaf growth rate were not carried out by the authors.

The analysis of plant physiological parameters and microclimate using wearable sensors was also described by Nassar and colleagues (npj Flexible Electron. 2018, 2(1), 1-12). Manufactured using a photolithographic process, the electrodes consisted of gold standards encapsulated between PDMS films (resistive voltage sensor for monitoring stem growth rate) or over PI/PDMS films (capacitive and resistive sensors integrated into leaves for humidity measurements and temperature, respectively). The thicknesses of the devices were around 200 μm (sensors for monitoring growth rate) and 54 μm (sensors for humidity and temperature measurements). However, as in the previous case, the authors did not carry out studies on the reproducibility of the sensors, biocompatibility and accuracy of electrical resistance data, which were associated with the growth of bamboo stems.

Assays to analyze leaf cellular water content were recently presented in the literature by Professor Trisha Andrew's group (Sci. Adv. 2019, 5(3), eaaw0463) from the University of Massachusetts. In this case, electrodes made of the polymer poly(3,4-propylenedioxythiophene) (PProDOT-CI) were printed directly onto leaves using physical masks and vapor phase polymerization. The electrodes were 5 μm thick. The samples were introduced into a reactor at a pressure of 1000 mTorr and were maintained at room temperature throughout the deposition process, which lasted approximately 20 min. A solid FeCl₃ target, positioned 15 cm from the leaf, was sublimated at 200° C., while the conductive polymer monomer was heated to 80° C. Vapor phase polymerization did not affect the health of the plants and the electrodes proved to be biocompatible based on the analysis of the parameters: phototropism, chlorophyll concentration (photosynthesis rate) and biomass generation (growth rate). The bioimpedance spectroscopy (EBI) technique was able to quantify cellular water content and reveal information about the composition of the cell wall. According to the equivalent RC circuit obtained to model the measurements in leaves, the electrical data were divided into two components depending on the frequency, namely, the electrode (<10³ Hz) and plant tissue (>10³ Hz). This last component reflects the health of the leaves and, in relation to its resistive parameters, the cell membrane capacitance (CM) values were the ones that generated the greatest sensitivity for quantifying water loss, calculated based on the gradual mass reduction of a leaf cut from its stem. This moisture stress was accelerated by inserting the leaf into a vacuum. Impedance and mass measurements were taken every 10 min over a 130 min interval. A 13% decrease in the leaf water content (77% to 64%) led to an approximately 70% drop in CM (˜0.3 nF change) which is presumably due to the loss of intracellular fluids rich in ionic nutrients, with a consequent decrease in the dielectric constant.

Despite the significant contributions of the work under discussion (Sci. Adv. 2019, 5(3), eaaw0463) to the use of the bioimpedance technique in the analysis of cellular water in leaves and for the establishment of sensor biocompatibility study protocols, some crucial limitations can be pointed out. Again, reproducibility and accuracy tests were not performed. Although biocompatibility tests were carried out, they were not based on the analysis of the leaves exposed to the electrodes, but on the roots of the plants, which does not allow an accurate inference about the real effect of the electrodes on the physiology of the leaf. Another obstacle concerns the manufacture of the sensor, with the insertion of the leaf into a reactor to record the electrodes directly on its surface. This method is especially unfeasible for testing a large number of samples. Thus, it can be stated that the real potential of wearable sensors for monitoring plant health has not yet been demonstrated in the literature with adequate statistical robustness.

### SUMMARY OF THE INVENTION

It is one of the objectives of the present description to disclose a leaf-wearable sensor for analyzing cellular water by electrochemical impedance spectroscopy (EIE), wherein said sensor is provided with a flexible electrode, of adequate robustness for long-term tests in situ, presenting reproducibility during sensing and being biocompatible and non-harmful to leaf health. Another objective of the present description is to reveal a production process for said sensor that has reproducibility and scalability.

The objectives of the present description are achieved by a leaf-wearable sensor for analyzing cellular water by EIE comprising: reading and actuation means for EIE assays; at least two electrodes made of nickel (Ni) metallic films; at least two electrical contacts, connected to the Ni electrodes and the reading and actuation means; and means to fix the Ni electrodes on plant leaves in vivo and in situ. Said Ni electrodes present special characteristics due to their geometric pattern and manufacturing process, as disclosed herein in embodiments of the description and illustrated in the figures.

The objectives of the present description are also achieved by a process of producing said leaf-wearable sensor for analyzing cellular water by EIE, the process comprising steps of producing at least two Ni electrodes and their respective electrical contacts, which comprises: (i) patterning of a photoresist mold by photolithography, in which the mold is deposited on a glass substrate, coated with thin metallic films, preferably formed from layers of chromium (Cr) and gold (Au), (ii) electrodeposition of a Ni metallic film on this mold, and (iii) removal of the photoresist and the Cr/Au layer to obtain the patterned Ni electrodes and their electrical contacts.

### BRIEF DESCRIPTION OF DRAWINGS

The present invention is illustrated in the embodiments represented in figures, as briefly described below.

FIG. 1A is a photograph of the patterned mold, obtained after developing the photoresist parts not exposed to UV, according to an embodiment of the described process.

FIG. 1B is an image obtained by laser confocal microscopy of the Ni electrodes, obtained after the electrodeposition of this metal and the removal of the photoresist and the Cr/Au layers, according to one embodiment of the described process.

FIG. 1C is a photograph of an electrode fixed to an adhesive tape, according to an embodiment of the described sensor.

In FIG. 1D is a digital photograph of the main components of a sensing system, with its electrodes fixed to the epidermis of a soybean leaf, according to one embodiment of the described sensor.

FIG. 2A is a graph of the absorption spectrum in the UV-Vis region of an adhesive tape used to attach the electrodes to the leaves, according to one embodiment of the described sensor.

FIG. 2B is a laser confocal microscopy image highlighting the roughness of an adhesive tape used to attach the electrodes to the leaves, according to one embodiment of the described sensor.

FIG. 3 is a graph with the dehydration curve obtained for soybean leaves at an average temperature of 19.15±0.39° C. and relative air humidity of 45.40±10.86%, over 5 h, obtained with the use of an embodiment of the described sensor.

FIG. 4 is a typical Bode diagram of the sensing system, with curves plotted every 20 min during the soybean leaf dehydration process, conducted using one embodiment of the described sensor.

FIG. 5 is an analytical impedance curve as a function of dehydration of soybean leaves, conducted using an embodiment of the described sensor.

FIG. 6 is a series of graphs with analytical curves of impedance as a function of water loss from soybean leaves, at frequencies that generated the highest sensitivities, conducted using an embodiment of the described sensor.

FIG. 7A is a parity plot of true (experimentally measured) water loss values and calculated values of test samples, conducted using an embodiment of the described sensor.

FIG. 7B is a bar graph of accuracy data for the test samples of FIG. 7A, conducted using an embodiment of the described sensor.

### DETAILED DESCRIPTION OF THE EMBODIMENTS

The description refers to a leaf-wearable sensor for analyzing cellular water and the production process of this sensor.

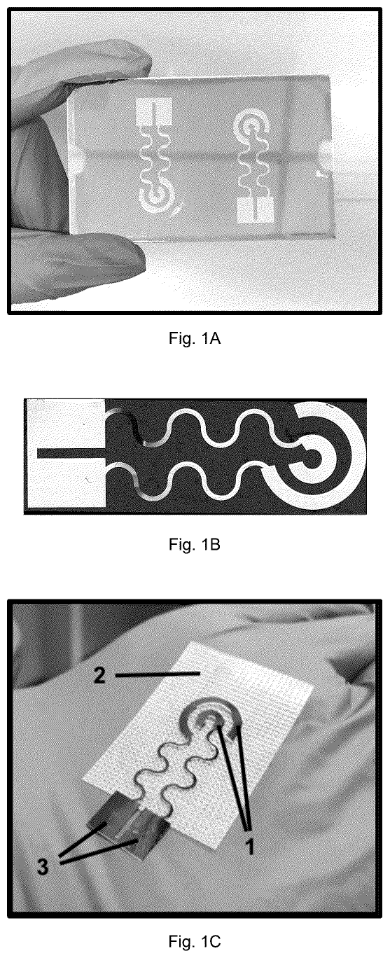

FIGS. 1A to 1C illustrate some steps in the production process of a sensor embodiment, while FIG. 1D shows a preferred sensor embodiment, worn in a soybean leaf.

In any embodiments, the sensor comprises:
Signal reading and actuation means (5) for carrying out electrochemical impedance spectroscopy (EIE) tests, preferably a portable commercial potentiostat, favoring the application of the sensor in situ;
at least two flexible, patterned Ni electrodes (1), made of stand-alone metallic films (stand-alone meaning free and suspended, without the need to be supported by a substrate);
at least two electrical contacts (3) electrically connected to the Ni electrodes (1) and the signal reading and actuation means (5); and
fixing means (2) of Ni electrodes on plant leaves in vivo, being made of flexible, porous and transparent material.

In any embodiment, the process comprises:
(i) patterning of a photoresist mold by photolithography, in which the mold is deposited on a glass substrate, coated with thin metallic films, preferably formed from layers of chromium (Cr) and gold (Au),
(ii) electrodeposition of a Ni film onto this mold, and
(iii) removal of the photoresist and the Cr/Au layer to obtain the patterned Ni electrodes (1) and the electrical contacts (3) electrically connected to the Ni electrodes (1);
(iv) fixing the Ni electrodes (1) in fixing means (2), the fixing means (2) being made of flexible, porous and transparent material;
(v) electrical connection of signal reading and actuation means (5) with the electrical contacts (3).

In one embodiment of the described process, the patterned mold is manufactured on a glass substrate. The substrate is coated with thin films of Cr and Au, preferably a 25 nm thick Cr film followed by a 200 nm thick Au film, both deposited using sputtering coating technique. The substrate is heated to remove moisture, preferably at 120° C. for 10 min. Then, the photoresist is deposited on the substrate, preferably using the spin-coating for the deposition of hexamethyldisilazane (HMDS, 4000 rpm for 30 s) and AZ50XT (2000 and 3400 rpm for 20 and 5 s, respectively). The mold is then subjected to drying processes to eliminate solvent, preferably using pre-bake drying technique of HMDS films (120° C. for 10 min) and AZ50XT (heating ramp from 50 to 112° C. over 30 min). Subsequently, the photoresist is patterned by exposure to ultraviolet (UV) light in a photoaligner, preferably for 150 s at a intensity of 9.5 mW cm⁻² for generating electrode and contact patterns using the photoresist, using a mask obtained by direct laser engraving. Finally, the development step is carried out to remove the photoresist parts exposed to UV. FIG. 1A shows the mold obtained after revealing the photoresist parts not exposed to UV, according to this embodiment.

In one embodiment of the described process, after obtaining the mold, Ni electrodeposition is carried out in an electrochemical electroplating bath. The bath uses the Cr/Au film present in the mold as the cathode, commercial nickel as the anode, and a direct current potential source applied between the cathode and the anode, preferably a current of 100 mA for 90 min. After Ni electrodeposition, the photoresist is removed by immersing the mold in acetone and then the Cr and Au layers are removed with their respective etching solutions. Finally, the Ni film comprising patterned Ni electrodes (1) and electrical contacts (3) is mechanically removed from the glass substrate, for example with the aid of a scalpel, washed with Extran® and isopropanol and dried under nitrogen flow.

FIG. 1B shows an image obtained by laser confocal microscopy of the Ni film obtained by an embodiment of the present process. The Ni film is a stand-alone integral structure, such a structure comprising Ni electrodes (1) electrically connected to electrical contacts (3) by Ni tracks.

In one embodiment of the described process, the Ni film obtained has a thickness of 30 to 50 μm, being flexible, bendable, with high mechanical stability.

In one embodiment, the Ni film obtained has two Ni electrodes (1) in the form of concentric semicircles, having diameters of 5500 and 4000 μm, respectively, both 15 μm wide and 40 μm thick. This shape is designed in order to optimize the geometric area of the electrode, maximizing the length of the electrode and minimizing the footprint of the leaf width.

In one embodiment, the Ni film obtained presents tracks in the form of sinusoidal curves electrically connected the Ni electrodes (1) to the electrical contacts (3), forming an extensible pattern that provides greater mechanical stability to the wearable sensor on the leaf.

In one embodiment, each electrical contact (3) is 500 μm wide, 800 μm long and 40 μm thick.

In one embodiment, the electrical connection of the signal reading and actuation means (5) with the patterned electrical contacts (3) is made by fixing the terminal of a flat cable on the electrical contacts (3) using a soldering iron and a 60:40 m/m lead and tin alloy. This solder is sufficient to withstand external disturbances, such as movement, humidity and temperature, inherent to the sensing of plant leaves in situ, without generating instability in the signal obtained by electrochemical impedance spectroscopy using the sensor described here.

In one embodiment, after the process of obtaining the Ni film in a stand-alone structure, additional steps are carried out to coat both sides of the Ni film with protective metallic layers, which are less susceptible to oxidation. Such protective metallic layers are intended to increase the durability of the sensor produced from the Ni film, since Ni oxidation can cause noise in the electrical readings of this sensor.

In one embodiment, to coat the Ni film with protective metallic layers of Au and Cu, the Ni film is fixed to a glass substrate using hexamethyldisilazane adhesion promoter and AZ50XT photoresist, using the spin-coating technique, preferably the HMDS at 4,000 rpm for 30 s and the AZ50XT in successive steps of 2,000 and 3,400 rpm for 20 and 5 s, respectively. The substrate is again subjected to drying processes to eliminate solvent, preferably using pre-bake drying technique of HMDS films (preferably at 120° C. for 10 min) and AZ50XT (preferably with a heating ramp from 50 to 112° C. over 30 min). Subsequently, Cr and Au are deposited using the sputtering technique, preferably with the respective thicknesses of Cr of 150 Å and Au of 1050 Å. Finally, the photoresist is removed with an appropriate solvent and the coated Ni film is removed using a blade. The coating process is repeated for both surfaces of the Ni film.

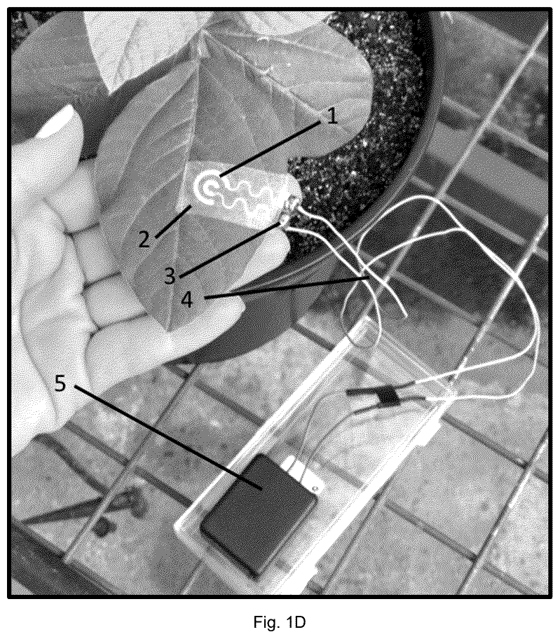

FIG. 2C shows an embodiment of Ni electrodes (1) fixed to fixing means (2) consisting of a transparent polymeric adhesive tape.

In turn, FIG. 2D shows the main elements of an embodiment of sensor, with the Ni electrodes (1) fixed to a soybean leaf by a transparent polymeric adhesive tape, which constitutes the fixing means (2) of this embodiment.

### Exemplary Uses of the Sensor of the Invention

Following, exemplary realizations of the use of the object described here are presented, in a non-restrictive sense, illustrating results and advantages achieved thereof.

In the embodiments of the following examples, the electrodes are attached to the leaves using transparent polystyrene adhesive tapes, commercially named Traspore Nexcare 3M® (provided by the company 3M DO BRASIL, Brazil). To verify the optical and porosity properties of this adhesive tape, spectroscopy analyzes were carried out in the UV-Vis region and confocal laser microscopy, represented in FIGS. 2A and 2B, respectively. The images made it possible to observe that the tape has standard structures of holes and low reliefs in different thicknesses, which guarantee strong adhesion with the leaves. The absorbance spectrum showed no peaks in the visible light region (400-700 nm), demonstrating the optical transparency of the tape. Therefore, due to its morphological characteristics and optical transparency, the tape is biocompatible as it does not impede the main metabolic functions of plants, namely respiration and photosynthesis.

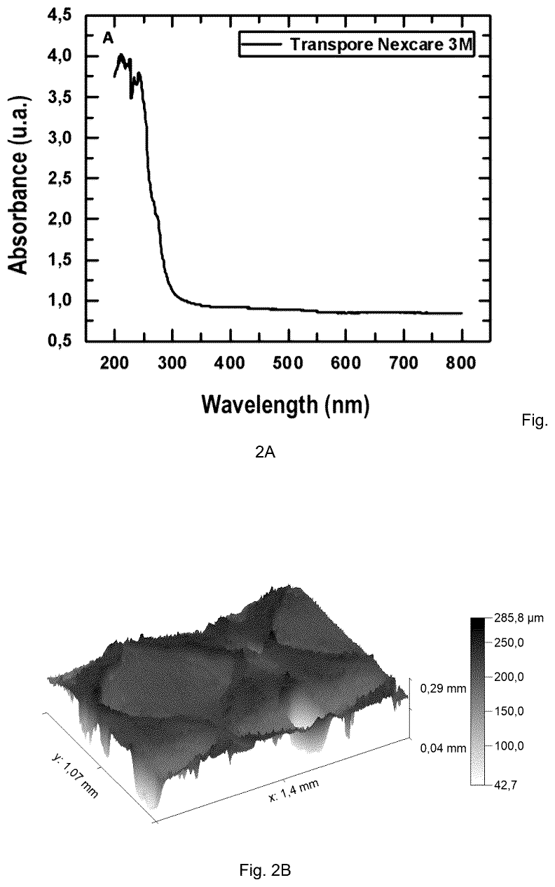

FIG. 3 shows a dehydration curve obtained from tests with an embodiment of sensor fixed to soybean leaves (n=20), conducted at a temperature in the range of 19.15±0.39° C. and relative air humidity in the range of 45.40±10.86%, for 5 hours. In the aforementioned temperature and humidity ranges, the dehydration rate was 0.38% min⁻¹ for the first 30 min of testing and 0.11% min⁻¹ from 30 min to 300 min.

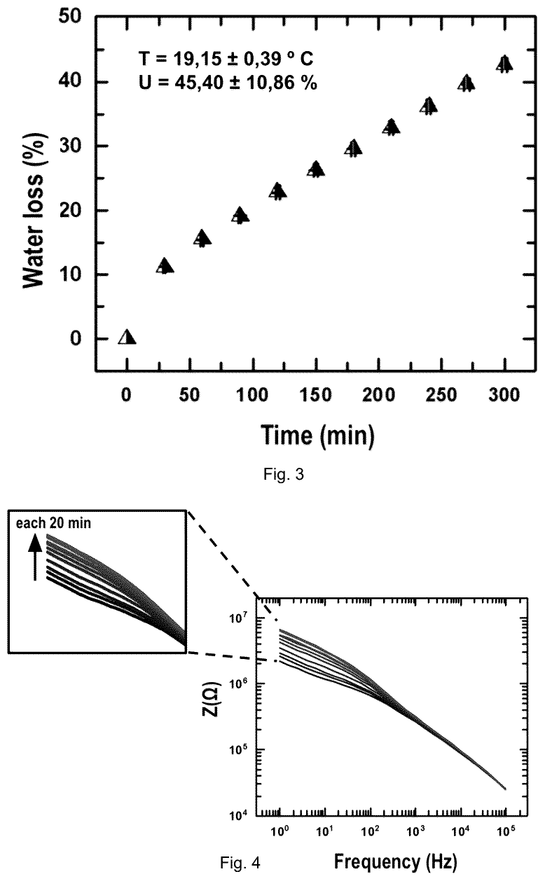

In FIG. 4, a typical Bode diagram of the system is presented. EIE measurements taken at 20 min intervals show an increase in impedance (Z) as leaf dehydration occurs. This result is due to the reduction in the diffusion of ions present in the aqueous leaf content to the leaf epidermis when polarized by the application of the potential. This response is more sensitive at lower frequencies, a region in which interface loading governs the sensor response.

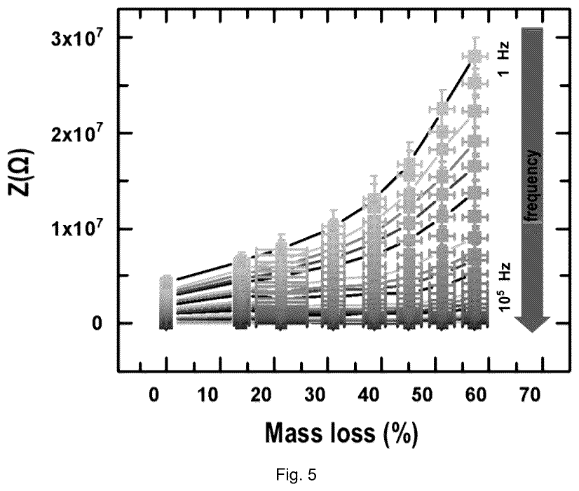

FIG. 5 presents analytical impedance curves as a function of dehydration of soybean leaves (n=20) for various frequencies from 100 to 105 Hz, in tests conducted at temperatures in the range of 19.15±0.39° C. and relative air humidity in the range of 45.40±10.86%. The spectra were obtained by applying a potential of 250 mV of alternating current.

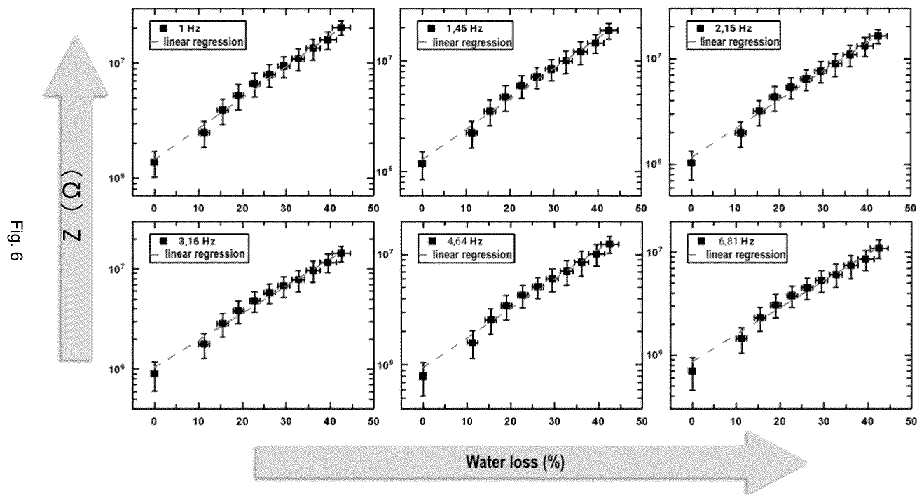

FIG. 6 presents a series of analytical impedance curves as a function of water loss from soybean leaves at frequencies that generated the highest sensitivities in the tests represented in FIG. 5.

In Table 1, values of the coefficients of determination (R²), sensitivity and detection limits (LD) for the frequencies that generated the highest sensitivities.

TABLE 1
Parameters obtained by linear adjustments of the analytical impedance
curves as a function of water loss from soybean leaves (n = 20).

| Frequency (Hz) | R² | Sensitivity | LD (Ω %⁻¹) |
|---|---|---|---|
| 1 | 0.9928 | 0.0269 | 9.91 × 10⁷ |
| 1.45 | 0.9918 | 0.0275 | 9.24 × 10⁷ |
| 2.15 | 0.9902 | 0.0273 | 8.62 × 10⁷ |
| 3.16 | 0.9938 | 0.0271 | 7.98 × 10⁷ |
| 4.64 | 0.9860 | 0.0267 | 7.52 × 10⁷ |
| 6.81 | 0.9823 | 0.0262 | 7.02 × 10⁷ |

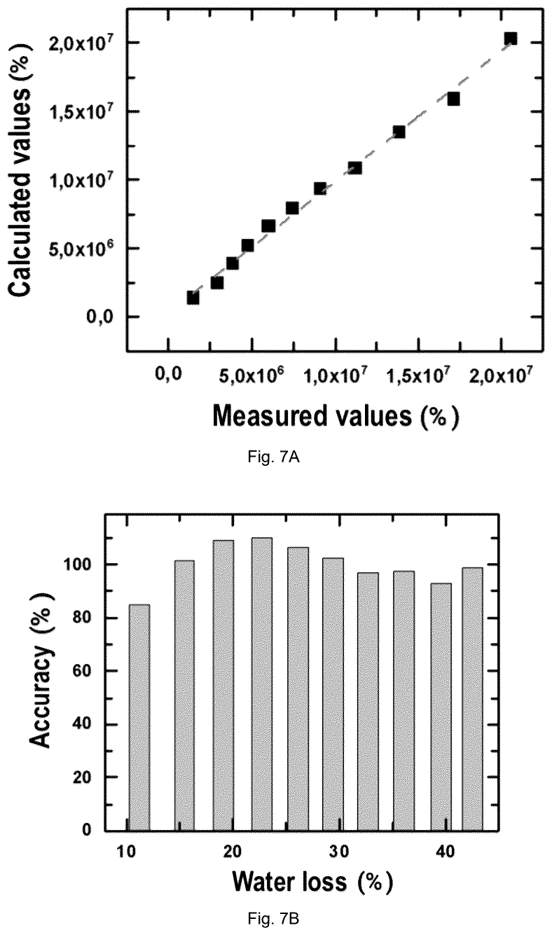

FIG. 7A is a parity plot of the true (experimentally measured) and calculated water loss values of the test samples. Such calculated values were obtained from the equation of the straight line of the analytical curve with the calibration samples. It is possible to verify that the values are close, demonstrating the sensor's ability to accurately quantify water loss from soybean leaves. In FIG. 7B, a bar graph of the accuracy for these test samples is displayed.

The present description revealed sensors comprising flexible, bendable electrodes, with high mechanical stability, scalable and stand-alone type with a reduced metal area, which contributes to its biocompatibility and adhesion on leaves. More specifically, the respiration and photosynthesis processes of the leaves are little affected by the sensor, generating good biocompatibility. Additionally, strong sensor adhesion is achieved, attributed to the high leaf/adhesive contact area, considering the preferred adhesive described herein in the exemplary embodiments. It should also be noted that this adhesive proved to be biocompatible due to its optical transparency and porous structure.

This invention allows EIE data to be correlated with the relative water content present in the leaf, which aims to monitor different types of stress of the plant, such as moisture and saline stresses, in addition to the action of toxic agents. This sensor can assist in fundamental studies (for example, tests associated with the development of agricultural products against the action of pests) and in precision agriculture itself through the use of portable EIE equipment and with connectivity to transmit data to a control center.

Although exemplary embodiments of the processes and products described have been presented in this application, the scope of protection is not intended to be limited to their literality. Therefore, the description should be interpreted not as limiting, but merely as exemplifications of particular embodiments that encompasses the inventive concept presented here. A person skilled in the art can readily apply teachings presented herein to analogous solutions arising therefrom, limited only by the scope of the claims of this application.

1. Leaf-wearable sensor for analyzing cellular water by electrochemical impedance spectroscopy, characterized by comprising:
signal reading and actuation means (5) for carrying out electrochemical impedance spectroscopy tests,
at least two Ni electrodes (1) made of stand-alone nickel metallic films, with a thickness of 30 to 50 μm;
at least two electrical contacts (3), electrically connected to respective Ni electrodes (1) and to the signal reading and actuation means (5); and
fixing means (2) to fix Ni electrodes on plant leaves in vivo, made of flexible, porous and transparent material.

2. Sensor, according to claim 1, characterized by the fact that the signal reading and actuation means (5) is a portable commercial potentiostat.

3. Sensor, according to any one of claims 1 to 2, characterized by the fact that the Ni electrodes (1) are arranged in the form of concentric semicircles.

4. Sensor, according to any one of claims 1 to 3, characterized by the fact that the Ni electrodes (1) are electrically connected to the electrical contacts (3) by tracks in the form of sinusoidal curves, and in which the Ni electrodes Ni (1), the electrical contacts (3) and the tracks are formed by a solid stand-alone Ni structure, with a thickness of 30 to 50 μm.

5. Sensor, according to any one of claims 1 to 4, characterized by the fact that the fixing means (2) is an adhesive tape formed by a transparent polymeric film.

6. Sensor, according to any one of claims 1 to 4, characterized by the fact that the Ni electrodes (1) are coated on both sides with protective metallic layers.

7. Sensor, according to claim 6, characterized by the fact that the protective metallic layers comprise a Cr layer and an Au layer, preferably with thicknesses Cr 150 Å and Au 1050 Å.

8. Production process for the sensor of claims 1 to 7, characterized in that it comprises:
(i) patterning of a photoresist mold by photolithography, in which the mold is deposited on a glass substrate coated with a layer of metallic thin films,
(ii) electrodeposition of a Ni film onto this mold, and
(iii) removal of the photoresist and the layer of metallic thin films to obtain a one-piece stand-alone Ni structure, comprising the Ni electrodes (1) and the electrical contacts (3);
(iv) fixing the Ni electrodes (1) in fixing means (2) made of flexible, porous and transparent material;
(v) electrical connection of signal reading and actuation means (5) with the electrical contacts (3).

9. Production process according to claim 8, characterized by the fact that the substrate is coated with thin films of Cr and Au, preferably a 25 nm thick Cr film followed by a 200 nm thick Au film. thickness, both deposited using a sputtering.

10. Production process, according to any one of claims 8 to 9, characterized by the fact that the photoresist is deposited on the substrate by the technique of spin-coating.

11. Production process, according to any one of claims 8 to 10, characterized by the fact that the Ni film is deposited on the photoresist by electrochemical electroplating bath, using the thin metallic film present in the mold as cathode, commercial nickel as anode, and a direct current potential source applied between the cathode and the anode.

12. Production process, according to any one of claims 8 to 11, characterized by the fact that the removal of the photoresist and layers of metallic thin films is carried out by immersing the mold in acetone and then in solutions of etching suitable for the respective metallic thin films.
